(19) United States
(12) Patent Application Publication
(10) Pub. No.: US 2008/0096866 A1
(43) Pub. Date: Apr. 24, 2008

(54) Complex Formulation Of 3-Hydroxy-3-Methyl Glutaryl Coa Reductace Inhibitor And Antihypertensive Agent, And Process For Preparing Same

(75) Inventors: Jong Soo Woo, Gyeonggi-do (KR); Moon Hyuk Chi, Gyeonggi-do (JP); Yong II Kim, Gyeonggi-do (JP)

Correspondence Address: SUGHRUE MION, PLLC, 2100 PENNSYLVANIA AVENUE, N.W., SUITE 800, WASHINGTON, DC, 20037, US

(73) Assignee: Hanmi Pharm. Co., Ltd., Hwaseong-gun (KR)

(21) Appl. No.: 11/722,393

(22) PCT Filed: Dec. 28, 2005
(86) PCT No.: PCT/KR05/04607

§ 371 Date: Jun. 21, 2007

(30) Foreign Application Priority Data
Dec. 30, 2004 (KR) 10-2004-0116328

(51) Int. Cl. A61K 31/404; A61K 31/351; A61K 31/435; A61K 31/4439; A61P 9/12; A61K 31/554; A61K 31/44; A61K 31/40

(52) U.S. Cl. 514/211.09; 514/277; 514/338; 514/356; 514/419; 514/423; 514/460

(57) ABSTRACT

A complex formulation for oral administration comprising a sustained release formulation of an HMG-CoA reductase inhibitor and a film layer for rapid release of an anti-hypertensive agent, the film layer being coated on the sustained release formulation, can achieve improved therapeutic effects of the anti-hypertensive agent by promptly releasing it, while maintaining a constant drug level of the HMG-CoA reductase inhibitor in blood through a slow release. Accordingly, the complex formulation is useful for preventing and treating diseases such as hyperlipidemia, atherosclerosis, hypertension and cardiovascular disease.

### FIELD OF THE INVENTION

The present invention relates to a combination formulation for oral administration comprising a sustained release formulation of a 3-hydroxy-3-methylglutaryl coenzyme A (HMG-CoA) reductase inhibitor and a rapid release film layer of an anti-hypertensive agent; and a method for preparing the same.

### BACKGROUND OF THE INVENTION

Hypercholesterolemia, a representative example of hyperlipidemia, is caused by elevated serum LDL (low-density lipoprotein)-cholesterol and total cholesterol levels, and the treatment of hypercholestrolemia by reducing the level of lipid, especially LDL-cholesterol, in serum, makes it possible to lower the risk of cardiovascular disorders, which leads to delayed progression of arteriosclerosis (American diabetes association, Diabetic care, 23 (suppl.), S57-S65, 2000). Therefore, there have been many studies on lipid-lowering therapy for delaying the progression of arteriosclerosis or alleviating arteriosclerosis so as to reduce the risk of cardiovascular disorders, e.g., coronary heart disease, in a patient diagnosed as hyperlipidemia or hypercholestrolemia.

3-Hydroxy-3-methylglutaryl coenzyme A (HMG-CoA) reductase inhibitor used for hyperlipidemia such as hypercholesterolemia has been known to inhibit the conversion of HMG-CoA into mevalonate in the early stage of the cholesterol biosynthetic pathway, which results in lowering the total cholesterol and LDL-cholesterol levels, or elevating the high-density lipoprotein (HDL)-cholesterol level (S. M. Grundy, N. Engl. J. Med., 319(1), 24-32, 1988). However, such an HMG-CoA reductase inhibitor causes side effects such as liver toxicity, myopathy and rhabdomyolysis (Garnett W. R., Am. J. Cardiol., 78, 20-25, 1996; Dujovne C. A. et. al., Am. J. Med., 91, 25S-30S, 1991; and Mantell G. et. al., Am. J. Cardiol, 66, 11B-15B, 1990).

Accordingly, there have been numerous attempts to develop a sustained release formulation of an HMG-CoA reductase inhibitor in order to prevent or alleviate the side effects induced by the rapid release of HMG-CoA reductase inhibitor.

Many studies have suggested that a sustained release formulation of an HMG-CoA reductase inhibitor gives a lower bioavailability of the HMG-CoA reductase inhibitor for systemic circulation as compared with a rapid release formulation because most of the HMG-CoA reductase inhibitor absorbed into the body is metabolized in the liver (85% and more) while only 5% or less account for that transferred to the systemic circulation system. However, the drug delivering efficiency of a sustained release formulation to a target site is shown to be superior to that of a rapid release formulation (John R, Amer. J. Cardio. 89: 15, 2002). Accordingly, a sustained release formulation of an HMG-CoA reductase inhibitor has been reported to be more effective in lowering the LDL-cholesterol level in blood than a rapid release formulation (Monique P, Am. J. Drug Deliv. 1(4): 287-290, 2003).

Hypertension is accompanied by hyperlipidemia in many cases, which may cause cardiac disorders such as angina pectoris, and thus, it is very important to control hypertension together with administering an inhibitor of cholesterol-synthesis no matter whether or not the patent is suffering from coronary heart diseases, in order to reduce the risk or fatality arising from cardiovascular disorders.

For example, Kramsch et. al. have disclosed that a calcium channel blocking agent such as amlodipine, an antihypertension agent, can be administered together with a lipid-lowering agent to enhance the therapeutic effects against atherosclerosis (Kramsch et. al, Journal of Human Hypertension, Suppl. 1, 53-59, 1995), and Lichtlen P. R. et. al. have reported that early atherosclerotic disease in human can be effectively treated by administering with a calcium channel blocking agent (Lichtlen P. R. et. al., Lancet, 335, 1109-1139, 1990; and Waters D. et. al., Circulation, 82, 1940-1953, 1990).

Further, U.S. Pat. No. 4,681,893 disclosed that some statin drugs including atrovastatin are useful for treating atherosclerosis, and it has been reported that in case of administering a statin drug (pravastatin or lovastatin) together with a calcium channel blocking agent (amlodipine), atherosclerotic diseases can be better treated through the synergistic effects of the two drugs (Jukema et. al., Circulation, Suppl. 1, 1-197, 1995; and Orekhov et. al., Cardiovescular Drug and Therapy, 11, 350, 1997). However, Caduet® (Pfizer), a commercially available atrovastatin-amlodipine combination formulation, has the problem that both drugs are rapidly released causing liver toxicity, while therapeutic effects thereof cannot be maintained over a long period.

The present inventors have therefore endeavored to develop a combination formulation for oral administration of HMG-CoA reductase inhibitor and antihypertensive agent that is free from the above problems, and have found that a combination formulation for oral administration comprising a sustained release formulation of an HMG-CoA reductase inhibitor coated with a rapid release film layer of an anti-hypertensive agent exhibits unexpected synergistic effects of two drugs with minimal side effects.

### SUMMARY OF THE INVENTION

Accordingly, it is an object of the present invention to provide a combination formulation of an HMG-CoA reductase inhibitor and an anti-hypertensive agent, which exhibits synergic effects of two drugs with minimal side effects.

It is another object of the present invention to provide a method for preparing said formulation.

In accordance with one aspect of the present invention, there is provided a combination formulation comprising a sustained release formulation of an HMG-CoA reductase inhibitor and a rapid release film layer containing an anti-hypertensive agent, the rapid release film layer being coated on the sustained release formulation.

In accordance with another aspect of the present invention, there is provided a method for preparing the combination formulation, which comprises the steps of:

1) drying a mixture of a HMG-CoA reductase inhibitor, a solubilizing carrier and a stabilizing agent to obtain a solid dispersion;

2) dry-blending the solid dispersion obtained in step 1 with a carrier for sustained release and a gel hydration accelerator, and formulating the dry-blended mixture to obtain a sustained release formulation; and

3) coating the sustained release formulation obtained in step 2 with a rapid release film layer comprising the antihypertensive agent to obtain the combination formulation.

### BRIEF DESCRIPTION OF THE DRAWINGS

The above and other objects and features of the present invention will become apparent from the following description of the invention, when taken in conjunction with the accompanying drawings which respectively show:

FIG. 1: a cross-sectional diagram of a representative example of the inventive combination formulation;

FIG. 2: the solubilities of the solid dispersions prepared in Examples 1 to 3 and Comparative Example 1;

FIG. 3: the drug dissolution rates of the sustained release formulations prepared in Examples 4 to 6;

FIG. 4: the drug dissolution rates of the sustained release formulations prepared in Examples 7 and 8;

FIG. 5: the simvastatin dissolution rates of the combination formulations prepared in Examples 9 to 11;

FIG. 6: the simvastatin dissolution rate of the combination formulation prepared in Example 9 at a spin velocity of 50, 100 or 150 rpm; and

FIG. 7: the amlodipine dissolution rates of the combination formulations prepared in Examples 9 to 11, and Norvasc® (Pfizer).

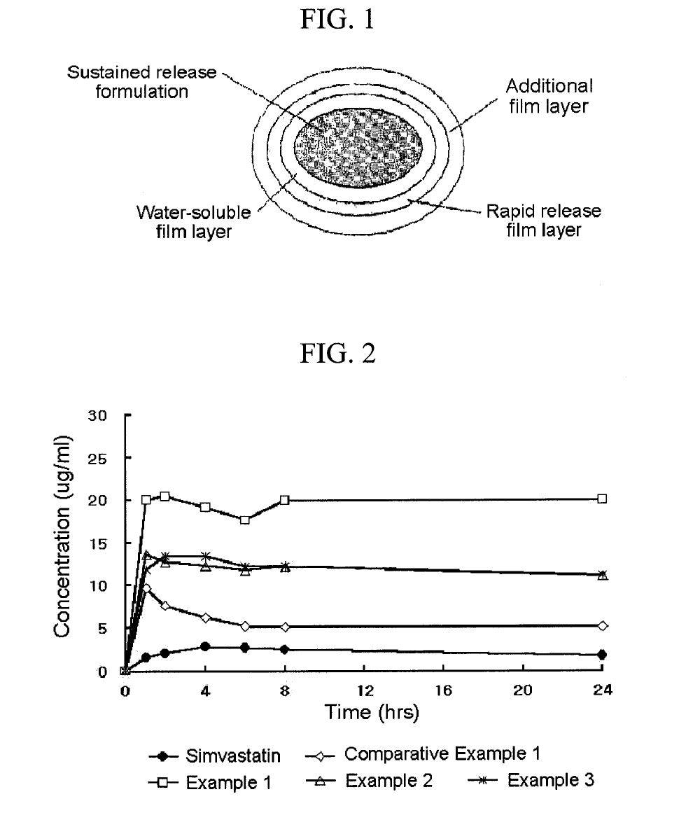

### DETAILED DESCRIPTION OF THE INVENTION

Hereinafter, the components of the combination formulation of the present invention are described in detail as follows:

1. Sustained Release Formulation

The sustained release formulation corresponding to the nucleus of the inventive combination formulation comprises a solid dispersion comprising an HMG-CoA reductase inhibitor as an active ingredient, solubilizing carrier and stabilizing agent; a carrier for sustained release; and a gel hydration accelerator.

1) Pharmacologically Active Ingredient

The HMG-CoA reductase inhibitor may be one of the known HMG-CoA reductase inhibitors used for treating hyperlipidemia and arteriosclerosis by lowering the lipoprotein or lipid level in blood. Representative examples thereof include mevastatin (U.S. Pat. No. 3,983,140), lovastatin (U.S. Pat. No. 4,231,938), pravastatin (U.S. Pat. Nos. 4,346,227 and 4,410,629), lactone of pravastatin (U.S. Pat. No. 4,448,979), velostatin, simvastatin (U.S. Pat. Nos. 4,448,784 and 4,450,171), rivastatin, fluvastatin, atrovastatin, cerivastatin and the like. The HMG-CoA reductase inhibitor may be employed in an amount ranging from 1 to 50% by weight, preferably from 2 to 30% by weight based on the total weight of the combination formulation. When the amount is less than 1% by weight, its therapeutic effect cannot be expected, and when more than 50% by weight, it exceeds the allowable daily dose.

2) Solubilizing Carrier

Since most HMG-CoA reductase inhibitors are poorly water-soluble compounds, a solubilizing carrier is used for enhancing the drug's solubility in the present invention. Representative examples of the solubilizing carrier include vitamin E TPGS (d-α-tocopheryl polyethylene glycol 1000 succinate: Eastman), polyoxyethylene stearic acid ester (e.g., Myrj: ICI), polyethylene glycol, hydroxypropylmethylcellulose (HPMC, viscosity: 3 to 15 cps), polyoxypropylene-polyoxypropylene block copolymer and the like. The solubilizing carrier may used in an amount ranging from 0.05 to 20 parts by weight, preferably 0.1 to 10 parts by weight based on 1 part by weight of the HMG-CoA reductase inhibitor. When the amount is less than 0.05 parts by weight, it is difficult to achieve the drug solubilization, and when more than 10 parts by weight, the sustained release of the drug cannot be expected.

3) Stabilizing Agent

The stabilizing agent used in the present invention may be any one of the known stabilizing agents which prevent the drug oxidation during the process of preparing the solid dispersion comprising the solubilizing carrier or forming the film layer comprising the antihypertension agent. Exemplary stabilizing agents include butylated hydroxy toluene (BHT), butylated hydroxy anisol (BHA), erythorbic acid, ascorbic acid, tocopherol and the like. The sustained release formulation of the present invention may comprise the stabilizing agent in an amount ranging from 0.001 to 3 parts by weight, preferably 0.002 to 2 parts by weight based on 1 part by weight of the HMG-CoA reductase inhibitor. When the amount is less than 0.002 parts by weight, it is difficult to attain the expected drug stability, and when more than 3 parts by weight, the stability of the stabilizing agent itself becomes poor. Further, the film layer comprising the antihypertension agent may comprise the stabilizing agent in an amount ranging from 0.004 to 6 parts by weight, preferably 0.008 to 4 parts by weight based on 1 part by weight of the antihypertension agent. When the amount is less than 0.004 parts by weight, the desired drug stability cannot be achieved, and when more than 6 parts by weight, it is difficult to form the film layer.

4) Carrier for Sustained Release

In the present invention, a carrier for sustained release is used for forming a hydrogel and it is preferably a mixture of xanthan gum and locust bean gum. Generally, xanthan gum contributes to the structural integrity maintenance of the formulation, thereby minimizing the change in the dissolution rate by physical forces such as gastrointestinal motility, and locust bean gum enhances the structural integrity in combination with xanthan gum. If the carrier is a mixture of components having specific component ratio, the initial burst release and the change in dissolution rate caused by physical forces can be reduced.

The carrier for sustained release may be employed in an amount ranging from 0.5 to 20 parts by weight, preferably 1 to 10 parts by weight based on 1 part by weight of the HMG-CoA reductase inhibitor. When the amount is less than 0.5 parts by weight, the sustained release of the drug becomes unsatisfactory, and when more than 20 parts by weight, the drug may be released too slowly. Further, in case of using a mixture of xanthan gum and locust bean gum as the carrier for sustained release, locust gum may be used in an amount ranging from 0.01 to 5 parts by weight, preferably 0.05 to 2 parts by weight based on 1 part by weight of the xanthan gum.

5) Gel Hydration Accelerator

When the sustained release formulation of the present invention is brought into contact with in vivo aqueous medium, the gel hydration accelerator allows water to rapidly infiltrate into the internal core of the formulation through rapid hydration leading to the formulation of a single homogeneous gelated core. In the present invention, the gel hydration accelerator may be preferably a mixture of propylene glycol alginate and hydroxypropylmethylcellulose (HPMC). The HPMC used therein preferably has a viscosity ranging from 4,000 to 100,000 cps.

The gel hydration accelerator may be used in an amount ranging from 0.1 to 20 parts by weight, preferably from 0.5 to 10 parts by weight based on 1 part by weight of the HMG-CoA reductase inhibitor. When the amount is less than 0.1 parts by weight, the gel hydration cannot be expected, and when more than 20 parts by weight, it is difficult to control the release rate of the drug. Further, propylene glycol alginate may be employed in an amount ranging from 0.05 to 20 parts by weight, preferably 0.1 to 10 parts by weight based on 1 part by weight of HPMC.

6) Pharmaceutically Acceptable Additive

The sustained release formulation of the present invention may further comprise at least one of the known pharmaceutically acceptable additives such as a dispersing agent, binder, lubricating agent, sweetening agent, excipient and the like, in order to prepare a solid formulation suitable for oral administration. Representative examples of the pharmaceutically acceptable additive may include polyvinylpyrrolidone (PVP), gelatin, hydroxypropyl cellulose, sucrose fatty acid ester, talc, light anhydrous silicic acid, zinc and magnesium salts of stearic acid and the like.

2. Rapid Release Film Layer

The rapid release film layer of the present invention comprises an antihypertension agent as an active ingredient, which may be selected from the group consisting of calcium channel blocking agents such as amlodipine, isradipine, lacidipine, nicardipine, nifedipine, felodipine, nisoldipine, verapamil, diltiazem and mibefradil; beta blocking agents such as atenolol, metoprolol, bucidolol and carvediol; angiotensin-converting enzyme (ACE) inhibitors such as enalapril, fosinopril, lisinopril, perindopril, benazepril, captopril, trandolapril, losartan, irbesartan, candesartan, valsartan, telmisartan and eprosartan; and potassium-sparing agent such as amiloride and bendroflumethiazide. The antihypertension agent may be employed in an amount ranging from 0.5 to 30% by weight, preferably 1 to 20% by weight based on the weight of the inventive combination formulation. When the amount is less than 0.5% by weight, its therapeutic effect cannot be expected, and when more than 30% by weight, it is difficult to form the film layer.

The rapid release film layer of the present invention may comprise the stabilizing agent which is used for preparing the sustained release formulation, in order to prevent oxidation of the antihypertension agent. In the rapid release film layer, the stabilizing agent may be used in an amount ranging from 0.04 to 6 parts by weight based on the antihypertension agent.

Further, the rapid release film layer may comprise at least one of the known film-forming materials such as hydroxypropylmethylcellulose (HPMC), hydroxypropylcellulose (HPC), hydroxyethylcellulose (HEC), celluloseacetatephthalate (CAP), ethylcellulose (EC), methylcellulose (MC), polymethacrylate, Kollicoat® (Basf) and Opadry® (Colorcon). It may further comprise plasticizers such as polyethyleneglycol (PEG), glycerol triacetate (triacetine) and acetylated monoglyceride (Myvacet), and conventional solvents capable of dissolving the film-forming materials such as purified water or ethanol may be used to form the film layer.

3. Water-Soluble Film Layer

The inventive combination formulation may further comprise a water-soluble film layer disposed between the sustained release formulation and the rapid releasing film layer, which blocks the mutual contact of the HMG-CoA reductase inhibitor in the sustained release nucleus with the antihypertension agent in the rapid releasing film layer. The water-soluble film layer may be employed in an amount ranging from 0.5 to 20% by weight, preferably 1 to 10% by weight based on the weight of the inventive combination formulation. When the amount is less than 0.5% by weight, the blocking effect becomes unsatisfactory, and when more than 20% by weight, it adversely affects the drug release.

Further, the water-soluble film layer may comprise at least one of the known water-soluble film-forming materials such as hydroxypropylmethylcellulose (HPMC), hydroxypropylcellulose (HPC), hydroxyethylcellulose (HEC), celluloseacetatephthalate (CAP), ethylcellulose (EC), methylcellulose (MC), polymethacrylate, Kollicoat® (Basf) and Opadry® (Colorcon). It may further comprise plasticizers such as polyethyleneglycol (PEG), glycerol triacetate (triacetine) and acetylated monoglyceride (Myvacet), and conventional solvents capable of dissolving the film-forming materials such as purified water or ethanol may be used to form the film layer.

4. Additional Film Layer

The inventive combination formulation may further comprise an additional film layer on the outside of the rapid releasing film layer for the protection of the drugs from unfavorable factors such as light and moisture, as well as for the convenience of administration (e.g., masking bitterness). The additional film layer may be a light-shielding film layer, moisture-proof film layer or sugar film layer, which may be employed in an amount ranging from 0.5 to 20% by weight, preferably 1 to 10% by weight based on the weight of the inventive combination formulation. When the amount is less than 0.5% by weight, its protecting effect cannot be achieved, and when more than 20% by weight, it adversely affects the drug release.

Further, the additional film layer may comprise at least one of the known film-forming materials such as hydroxypropylmethylcellulose (HPMC), hydroxypropylcellulose (HPC), hydroxyethylcellulose (HEC), celluloseacetatephthalate (CAP), ethylcellulose (EC), methylcellulose (MC), polymethacrylate, Kollicoat® (Basf) and Opadry® (Colorcon). It may further comprise plasticizers such as polyethyleneglycol (PEG), glycerol triacetate (triacetine) and acetylated monoglyceride (Myvacet), and conventional solvents capable of dissolving the film-forming materials such as purified water or ethanol may be used to form the film layer.

The inventive combination formulation for oral administration of a HMG-CoA reductase inhibitor and an antihypertension agent may be prepared by the following steps:

1) drying a mixture of a HMG-CoA reductase inhibitor, a solubilizing carrier and a stabilizing agent to obtain a solid dispersion;

2) dry-blending the solid dispersion obtained in step 1 with a carrier for sustained release and a gel hydration accelerator, and formulating the dry-blended mixture to obtain a sustained release formulation; and

3) coating the sustained release formulation obtained in step 2 with a rapid release film layer comprising the antihypertensive agent to obtain the combination formulation.

In step 1, the solid dispersion may be prepared by a conventional method such as spray-drying, solvent evaporating, micropulverizing-wetting, melting, and freeze-drying methods, and may preferably have a particle size ranging from 5 to 200 μm in diameter. Further, the pharmaceutically acceptable additive as described above may be added to the solution for facilitating the formulation of the solid dispersion.

In step 2, the sustained release formulation may be formulated into a tablet by compressing the dry-blended mixture through direct compression, or by compressing, milling and tabletting the dry-blended mixture. Further, the blended mixture may further comprise a pharmaceutically acceptable additive for facilitating the formulation.

The above method may further comprise the step of coating the sustained release formulation obtained in step 2 with a water-soluble film layer before coating with the rapid release film layer in step 3.

Further, the above method may further comprise the step of coating the finally obtained combination formulation with an additional film layer for protecting the formulation from degenerative factors such as light and moisture as well as for enhancing the patient compliance (e.g., by blocking a bitter taste).

The oral combination formulation of the present invention comprising an HMG-CoA reductase inhibitor and an antihypertension agent have advantages in that it maximizes the therapeutic effects of the drugs by the synergism arising from combining the drugs having different release patterns or dosages: the antihypertension agent is rapidly released to enhance its therapeutic effect and the HMG-CoA reductase inhibitor is slowly released at a uniform rate to maintain its blood concentration. Further, the inventive combination formulation may further comprise a separating layer so as to minimize the contact between the two unstable constituent drugs. Accordingly, the inventive formulation can be effectively used for preventing and treating hyperlipidemia, arteriosclerosis, hypertension, cardiovascular disease and the combined disease thereof when orally administered once per day at a single dose.

The following Examples are intended to further illustrate the present invention without limiting its scope.

### EXAMPLES 1 TO 3 AND COMPARATIVE EXAMPLE 1

### Preparation of Solid Dispersion

Simvastatin (Hanmi Fine Chemical Co., Ltd., Korea), MYRJ (ICI, USA), HPMC 2910 (viscosity: 3 to 15 cps, Shin-Etsu, Japan), BHT (UENO Fine Chemical, USA) and light anhydrous silicic acid (as a dispersing agent) were dissolved in a mixture of ethanol and dichloromethane according to the amounts described in Table 1, respectively, and each of the resulting mixtures was subjected to spray-drying to obtain a solid dispersion having an average particle size of 100 μm or below. The solid dispersions of Examples 1 to 3 and Comparative Example 1 obtained thus are shown in Table 1.

TABLE 1

| | | Comparative | | | |
|---|---|---|---|---|---|
| Component (mg/tablet) | | Example 1 | Example 1 | Example 2 | Example 3 |
| Acitve | Simvastatin | 20 | 20 | 20 | 20 |
| ingredient | | | | | |
| Solubilizing | MYRJ | x | 40 | 20 | 20 |
| carrier | MPMC 2910 | 10 | 10 | 10 | 5 |
| Stabilizing | BHT | x | 2 | 2 | 2 |
| agent | | | | | |
| Additive | Light anhydrous | 5 | 5 | 5 | 5 |
| | silicic acid | | | | |
| Solvent | Ethanol | 200 | 200 | 200 | 200 |
| | Dichloro Methane | 700 | 700 | 700 | 700 |

### EXAMPLES 4 TO 8

### Preparation of Sustained Release Formulation for Oral Administration

The procedure of Example 1 was repeated using Simvastatin, lovastatin or fluvastatin as an active ingredient, together with MYRJ, HPMC 2910, BHT, and light anhydrous silicic acid according to the amounts described in Tables 2 to 4, respectively, to obtain solid dispersions. Then, each of the solid dispersions was mixed with xanthan gum (Kelco, USA), locust bean gum (Cesalpinia, Italy), propylene glycol alginate (ISP, USA), HPMC 2208 (viscosity: 4,000 to 100,000 cps, Shin-Etsu, Japan) and erythorbic acid for about 30 min; and sucrose fatty acid ester and light anhydrous silicic acid powders (finer than mesh 40) were added thereto, and mixed for 5 min. Each of the resulting mixtures was mold into a mass using a shaping assembler, and the mass was crushed down into particles having a mesh size ranging from 20 to 80. The particles were then formulated into a tablet by compressing in a formulator, to obtain a sustained release formulation. The sustained release formulations of Examples 4 to 8 obtained thus are shown in Tables 2 to 4.

TABLE 2

| Component (mg/tablet) | | Example 4 | Example 5 | Example 6 |
|---|---|---|---|---|
| Active | Simvastatin | 10 | 20 | 40 |
| ingredient | | | | |
| Solubilizing | MYRJ | 20 | 20 | 20 |
| agent | HPMC | 10 | 10 | 19 |
| | 2910 | | | |
| Stabilizing | BHT | 2 | 2 | 2 |
| agent | Erythorbic | 15 | 15 | 15 |
| | Acid | | | |
| Gel | HPMC | 80 | 80 | 80 |
| hydration | 2208 | | | |
| accelerator | Propylene | 43 | 43 | 43 |
| | glycol alginate | | | |
| Carrier for | Xanthan gum | 80 | 80 | 80 |
| sustained | Locust bean | 10 | 10 | 10 |
| release | gum | | | |
| Additive | Light anhydrous | 25 | 25 | 25 |
| | silicic acid | | | |
| | Sucrose fatty | 10 | 10 | 10 |
| | acid ester | | | |

TABLE 3

| Component (mg/tablet) | | Example 7 |
|---|---|---|
| Active | Lovastatin | 60 |
| ingredient | | |
| Solubilizing | MYRJ | 20 |
| carrier | HPMC 2910 | 10 |
| Stabilizing | BHT | 2 |
| agent | Erythorbic acid | 15 |
| Gel hydration | HPMC 2208 | 80 |
| accelerator | Propylene glycol alginate | 43 |
| Carrier for | Xanthan gum | 80 |
| sustained release | Locust bean gum | 10 |
| Additive | Light anhydrous silicic acid | 25 |
| | Sucrose fatty acid ester | 10 |

TABLE 4

| Component (mg/tablet) | | Example 8 |
|---|---|---|
| Active | Fluvastatin | 60 |
| ingredient | | |
| Solubilizing | MYRJ | 20 |
| carrier | HPMC 2910 | 10 |
| Stabilizing | BHT | 2 |
| agent | Erythorbic acid | 15 |
| Gel | HPMC 2208 | 80 |
| hydration | Propylene glycol alginate | 43 |
| accelerator | | |
| Carrier for | Xanthan gum | 80 |
| sustained release | Locust bean gum | 10 |
| Additive | Light anhydrous silicic acid | 25 |
| | Sucrose fatty acid ester | 10 |

### EXAMPLES 9 TO 11

### Preparation of Combination Formulation for Oral Administration

Each of the sustained release formulations obtained in Examples 5, 7 and 8 was coated with Opadry® AMB (Colorcon) film. Amlodipine camsylate (Hanmi Fine Chemical Co., Ltd., Korea), HPMC 2910 (viscosity: 3 to 15 cps) and acetylated monoglyceride (Myvacet) were dissolved in a mixture of ethanol and dichloromethane according to the amounts described in Table 5, respectively, which was coated on the previous film-coated formulation.

TABLE 5

| Component (mg/tablet) | | Example 9 | Example 10 | Example 11 |
|---|---|---|---|---|
| Sustained formulation nucleus | | Example 5 | Example 7 | Example 8 |
| Active | Amlodipine | 7.9 | 7.9 | 7.9 |
| Ingredient | camsylate | | | |
| Coating | HPMC 2910 | 10 | 10 | 10 |
| Agent | | | | |
| Plasticizer | Myvacet | 1.6 | 1.6 | 1.6 |
| Stabilizing | BHT | 0.1 | 0.1 | 0.1 |
| Agent | | | | |
| Solvent | Ethanol | 120 | 120 | 120 |
| | Dichloro methane | 30 | 30 | 30 |

Each of the formulations thus obtained was further coated with a mixture prepared according to the composition described in Table 6 in order to protect amlodipine from light, to obtain a combination formulation. The combination formulations of Examples 9 to 11 are shown in Table 5. Titanium dioxide and HPMC 2910 were used for light-shielding, and polyethylene glycol 6000 (PEG 6000) and talc, as a plasticizer.

TABLE 6

| | Component | | | | | |
|---|---|---|---|---|---|---|
| | HPMC | Titatium | PEG | | | Distilled |
| | 2910 | dioxide | 6000 | Talc | Ethanol | water |
| mg/tablet | 8 | 1.6 | 1.2 | 0.3 | 80 | 30 |

### TEST EXAMPLE 1

### Solubility Test of Solid Dispersion

The solid dispersions of Comparative Example 1 and Examples 1 to 3, and a simvastatin powder as a control were each subjected to solubility test in distilled water using a dissolution-test system under the following conditions according to the 1^{st} Basket method described in Korea Pharmacopoeia.
Dissolution-test system: Erweka DT 80 (Erweka, Germany)
Effluent: 900 ml of distilled water
Temperature of effluent: 37±0.5° C.
Rotational speed: 50, 100 and 150 rpm
Analytic method: liquid chromatography
Column: Cosmosil C₁₈ (Nacalai tesque)
Mobile phase: acetonitrile/pH 4.0 buffer solution
Flow rate: 1.5 ml/min
Detector: ultraviolet spectrophotometer (238 nm)
Injection volume: 20 μl

The pH 4.0 buffer solution was prepared by mixing 3 ml of glacial acetic acid with 1 L of distilled water and adjusting the pH of the mixture to 4.0 with NaOH.

As can be seen in FIG. 2, the solid dispersions of Examples 1 to 3 showed higher solubilities as compared to the solid dispersion of Comparative Example 1 or the simvastatin powder, and the solubility seems to increase with the amount of MYRJ rather than that of HPMC.

### TEST EXAMPLE 2

### Dissolution Test of Sustained Release Formulation for Amount of Active Ingredient

The sustained release formulations prepared in Examples 4 to 6 were each subjected to drug dissolution test under the following conditions according to the 2^{nd} Paddle method described in Korea Pharmacopoeia. The amount of simvastatin eluted from the formulation during the test was measured by liquid chromatography at 1, 2, 4, 6, 8, 10, 12, 16, 20 and 24 hrs after starting the test.
Dissolution-test system: Erweka DT 80
Effluent: 0.01 M sodium phosphate buffer solution (pH 7.0) containing 0.5% sodium lauryl sulfate (SLS)
Temperature of effluent: 37±0.5° C.
Rotational speed: 100 rpm
Analytic method: ultraviolet spectrophotometer (247 nm and 257 nm)
Calculation of eluted amount: Cumulative release amount

The sample harvested at each designated time was reacted with 40 mg of pre-washed MnO₂ (under USP Simvastatin Tablet 1) for 30 min and centrifuged at 3,000 rpm for 5 min. Then, the absorbances at 247 and 257 nm of each sample were measured using an ultraviolet spectrophotometer and the actual absorbance was calculated by subtracting the absorbance at 257 nm from that at 247 nm.

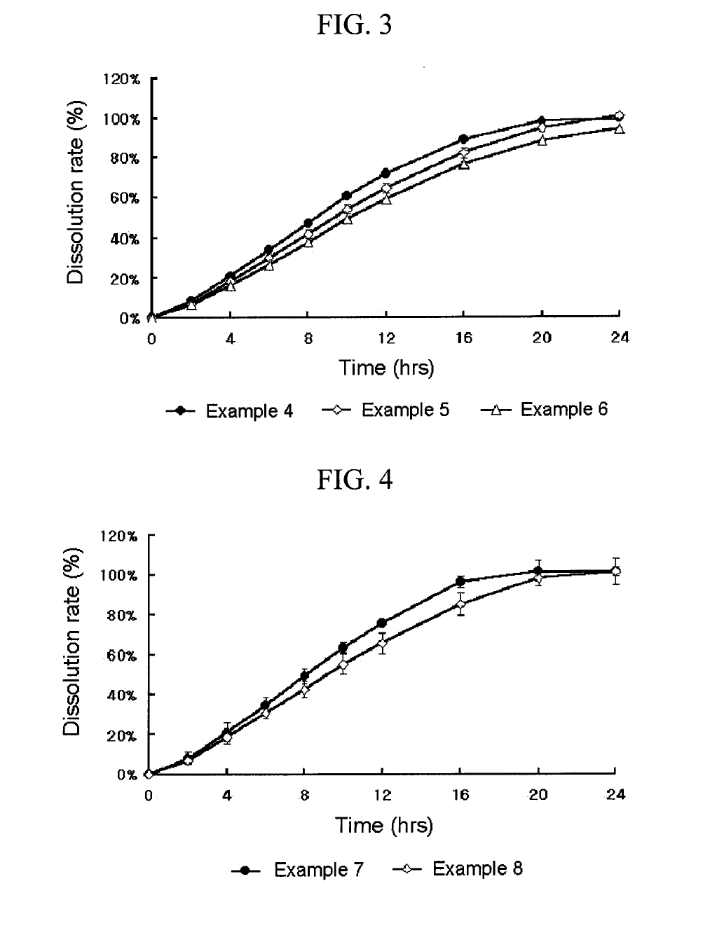

As shown in FIG. 3, the formulations obtained in Examples 4 to 6 exhibit similar dissolution rates regardless of the difference in the active ingredient content.

### TEST EXAMPLE 3

### Dissolution Test of Sustained Release Formulation for Kind of Active Ingredient

The formulations prepared in Examples 7 and 8 were subjected to dissolution test according to the same method as described in Test Example 2, except for measuring the amount of lovastatin or fluvastatin instead of that of simvastatin.

As a result, FIG. 4 shows that the sustained release formulations of Example 7 and 8 exhibit similarly sustained dissolution rates each other, irrespectively of kinds of the HMG-CoA reductase inhibitor used therein.

### TEST EXAMPLE 4

### Dissolution Test of Combination Formulation

The formulations prepared in Examples 9 to 11 were subjected to dissolution test according to the same method as described in Test Example 2, except for using a high performance liquid chromatograph (HPLC) instead of a ultraviolet spectrophotometer (UV) under the following conditions.
Dissolution-test system: Erweka DT 80
Effluent: 0.01 M sodium phosphate buffer solution (pH 7.0) containing 0.5% sodium lauryl sulfate (SLS)
Temperature of effluent: 37±0.5° C.
Rotational speed: 100 rpm
Analytic method: liquid chromatography
Column: Cosmosil C₁₈ (Nacalai tesque)
Mobile phase: acetonitrile/pH 4.0 buffer solution
Flow rate: 1.5 ml/min
Detector: ultraviolet spectrophotometer (238 nm)
Injection volume: 20 μl

The pH 4.0 buffer solution was prepared by mixing 3 ml of glacial acetic acid with 1 L of distilled water and adjusting the pH of the mixture to 4.0 with NaOH.

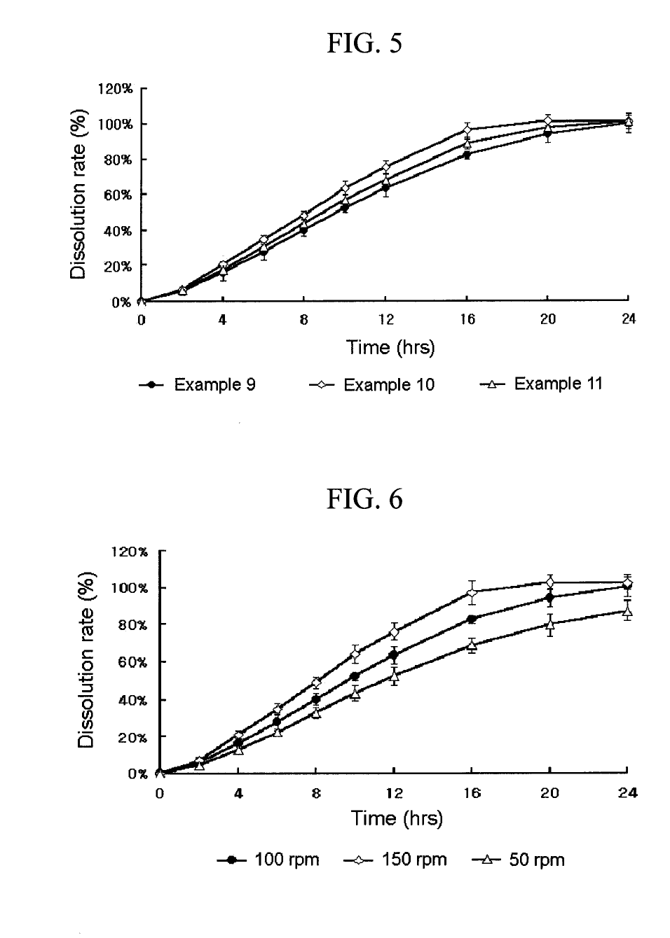

As shown in FIG. 5, the combination formulations of Examples 9 to 11 exhibit dissolution rates similar to those of the sustained release formulations of Examples 4 to 8. This suggests that the dissolution rate of the HMG-CoA reductase inhibitor is not much affected by the coating layers.

### TEST EXAMPLE 5

### Dissolution Test of Combination Formulation for Rotational Speed

The formulation prepared in Example 9 was subjected to dissolution test according to the same method as described in Test Example 4, except for setting the rotational speed at 50, 100 or 150 rpm.

As a result, FIG. 6 shows that the combination formulation of the present invention did not show any significant difference in the dissolution rates of the HMG-CoA reductase inhibitor even the rotational speed was changed. This suggests that side effects due to initial burst effect of the HMG-CoA reductase inhibitor would be significantly reduced when inventive combination formulation is administered to a patient.

### TEST EXAMPLE 6

### Dissolution Test of Combination Formulation

The combination formulations prepared in Examples 9 to 11, and the commercially available Norvasc® (Pfizer) as a comparative formulation were each subjected to a drug dissolution test under the following conditions according to the 2^{nd} Paddle method described in Korea Pharmacopoeia. The amount of amlodipine eluted from the test formulation during the test was measured by liquid chromatography at 15, 30, 45 and 60 min after starting the test.
Dissolution-test system: Erweka DT 80
Effluent: 500 ml of 0.01 N aqueous HCl
Temperature of effluent: 37±0.5° C.
Rotational speed: 75 rpm
Analytic method: ultraviolet spectrophotometer (237 nm)
Calculation of eluted amount: Cumulative release amount

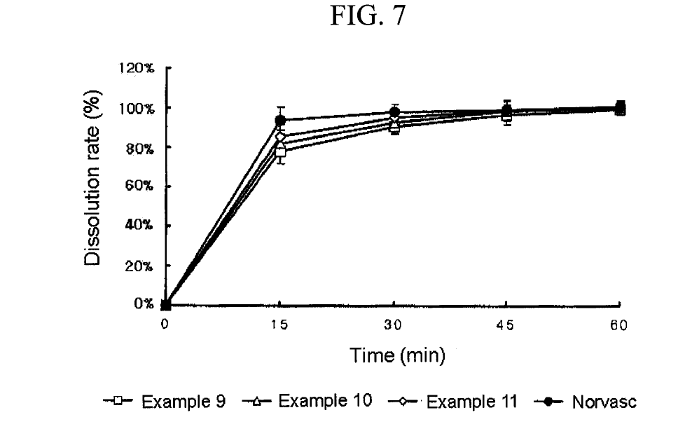

As shown in FIG. 7, the combination formulations of Examples 9 to 11 exhibit high amlodipine dissolution rates (90% at 30 min), similarly to the comparative formulation.

While the invention has been described with respect to the above specific embodiments, it should be recognized that various modifications and changes may be made to the invention by those skilled in the art which also fall within the scope of the invention as defined by the appended claims.

What is claimed is:

1. A combination formulation comprising a sustained release formulation of an HMG-CoA reductase inhibitor and a rapid release film layer containing an anti-hypertensive agent, the rapid release film layer being coated on the sustained release formulation.

2. The combination formulation of claim 1, wherein the sustained release formulation comprises a solid dispersant having the HMG-CoA reductase inhibitor, a solubilizing carrier and a stabilizing agent; a carrier for sustained release; and a gel hydration accelerator.

3. The combination formulation of claim 1, wherein the HMG-CoA reductase inhibitor is selected from the group consisting of mevastatin, lovastatin, pravastatin, lactone of pravastatin, velostatin, simvastatin, rivastatin, fluvastatin, atrovastatin and cerivastatin.

4. The combination formulation of claim 1, wherein the amount of the HMG-CoA reductase inhibitor ranges from 1 to 50% by weight based on the weight of the combination formulation.

5. The combination formulation of claim 2, wherein the solubilizing carrier is selected from the group consisting of vitamin E TPGS (d-α-tocopheryl polyethylene glycol 1000 succinate), polyoxyethylene stearate, hydroxypropylmethylcellulose, polyethylene glycol and polyoxypropylene-polyoxypropylene block copolymer.

6. The combination formulation of claim 2, wherein the amount of the solubilizing carrier ranges from 0.05 to 20 parts by weight based on 1 part by weight of the HMG-CoA reductase inhibitor.

7. The combination formulation of claim 2, wherein the stabilizing agent is selected from the group consisting of butylated hydroxy toluene (BHT), butylated hydroxy anisol (BHA), erythorbic acid, ascorbic acid and tocopherol.

8. The combination formulation of claim 2, wherein the amount of the stabilizing ranges from 0.001 to 3 parts by weight based on 1 part by weight of the HMG-CoA reductase inhibitor.

9. The combination formulation of claim 2, wherein the carrier for sustained release is a mixture of xanthan gum and locust bean gum.

10. The combination formulation of claim 9, wherein the amount of locust bean gum in the mixture ranges from 0.01 to 5 parts by weight based on 1 part by weight of the xanthan gum.

11. The combination formulation of claim 2, wherein the amount of the carrier for sustained release ranges from 0.5 to 20 parts by weight based on 1 part by weight of the HMG-CoA reductase inhibitor.

12. The combination formulation of claim 2, wherein the gel hydration accelerator is a mixture of propylene glycol alginate and hydroxypropylmethylcellulose (HPMC).

13. The combination formulation of claim 12, wherein HPMC has a viscosity ranging from 4,000 to 100,000 cps.

14. The combination formulation of claim 12, wherein the amount of the propylene glycol alginate ranges from 0.05 to 20 parts by weight based on 1 part by weight of HPMC.

15. The combination formulation of claim 2, wherein the amount of the gel hydration accelerator ranges from 0.1 to 20 parts by weight based on 1 part by weight of the HMG-CoA reductase inhibitor.

16. The combination formulation of claim 2, wherein the sustained release formulation further comprises a pharmaceutically acceptable additive.

17. The combination formulation of claim 16, wherein the pharmaceutically acceptable additive is selected from the group consisting of light anhydrous silicic acid, sucrose fatty acid ester, talc, zinc or magnesium salt of stearic acid, and a mixture thereof.

18. The combination formulation of claim 1, wherein the antihypertension agent is selected from the group consisting of amlodipine, isradipine, lacidipine, nicardipine, nifedipine, felodipine, nisoldipine, verapamil, diltiazem, mibefradil, atenolol, metoprolol, bucidolol, carvediol, enalapril, fosinopril, lisinopril, perindopril, benazepril, captopril, trandolapril, losartan, irbesartan, candesartan, valsartan, telmisartan, eprosartan, amiloride and bendroflumethiazide.

19. The combination formulation of claim 1, wherein the amount of the antihypertension agent ranges from 0.5 to 30% by weight based on the weight of the combination formulation.

20. The combination formulation of claim 1, wherein the rapid release film layer further comprises a stabilizing agent.

21. The combination formulation of claim 20, wherein the stabilizing agent is selected from the group consisting of butylated hydroxy toluene (BHT), butylated hydroxy anisol (BHA), erythorbic acid, ascorbic acid and tocopherol.

22. The combination formulation of claim 20, wherein the amount of the stabilizing agent ranges from 0.004 to 6 parts by weight based on 1 part by weight of the antihypertension agent.

23. The combination formulation of claim 1, which further comprises a water-soluble film layer disposed between the sustained release formulation and the rapid release film layer.

24. The combination formulation of claim 23, wherein the water-soluble film layer comprises at least one selected from the group consisting of hydroxypropylmethylcellulse (HPMC), hydroxy propylcellulose (HPC), hydroxyethylcellulose (HEC), celluloseacetatephthalate (CAP), ethylcellulose (EC), methylcellulose (MC), polymethacrylate, Kollicoat® (Basf) and Opadry® (Colorcon).

25. A method for preparing the combination formulation of claim 1, which comprises the steps of:
1) drying a mixture of a HMG-CoA reductase inhibitor, a solubilizing carrier and a stabilizing agent to obtain a solid dispersion;
2) dry-blending the solid dispersion obtained in step 1 with a carrier for sustained release and a gel hydration accelerator, and formulating the dry-blended mixture to obtain a sustained release formulation; and
3) coating the sustained release formulation obtained in step 2 with a rapid release film layer comprising the antihypertensive agent to obtain the combination formulation.

26. The method of claim 25, wherein the drying process of step 1 is conducted using a spray-drying method, a solvent evaporating method, a micropulverizing-wetting method, a melting method or a freeze-drying method.

27. The method of claim 25, wherein the solid dispersion obtained in step 1 has a particle size ranging from 5 to 200 μm in diameter.

28. The method of claim 25, wherein the formulating process of step 2 is conducted by directly compressing the dry-blended mixture to obtain a tablet, or by compressing, milling and tabletting the dry-blended mixture to obtain a tablet.

29. The method of claim 25, wherein step 1 further comprises the step of adding a pharmaceutically acceptable additive to the dry-blended mixture.

30. The method of claim 25, which further comprises the step of coating the sustained release formulation obtained in step 2 with a water-soluble film layer before coating with the rapid release film layer in step 3.

31. The method of claim 25, which further comprises the step of coating the combination formulation obtained in step 3 with an additional film layer.

32. The method of claim 31, wherein the additional film layer is a light-shielding film layer, moisture-proof film layer or sugar film layer.
